(19) United States
(12) Patent Application Publication
(10) Pub. No.: US 2016/0178590 A1
(43) Pub. Date: Jun. 23, 2016

(54) SYSTEM AND METHOD FOR PREDICTING HARMFUL MATERIALS

(71) Applicant: Electronics and Telecommunications Research Institute, Daejeon (KR)

(72) Inventors: Hyeon PARK, Daejeon (KR); Sung Soo KANG, Daejeon (KR); Hoon JEONG, Daejeon (KR)

(21) Appl. No.: 14/860,799

(22) Filed: Sep. 22, 2015

(30) Foreign Application Priority Data
Dec. 23, 2014 (KR) 10-2014-0187282

(51) Int. Cl. G01N 33/02

(52) U.S. Cl. CPC G01N 33/02

(57) ABSTRACT

Provided herein is a harmful material prediction system including a harmful material feature collecting unit configured to collect harmful material features of food; a preprocessing unit configured to preprocess the harmful material features collected and generate harmful material information of an analyzable format; and a Hadoop-based dispersed cluster configured to generate a similarity group where similarity base points are grouped based on a correlation per variable included in the harmful material information, thereby predicting in real time the harmful materials over the overall phases of distribution.

### CROSS-REFERENCE TO RELATED APPLICATION

The present application claims priority to Korean patent application number 10-2014-0187282, filed on Dec. 23, 2014, the entire disclosure of which is incorporated herein in its entirety by reference.

### BACKGROUND

1. Field of Invention

Various embodiments of the present invention relate to a system and method for predicting harmful materials, and more particularly, to a system and method for predicting harmful materials using big data.

2. Description of Related Art

In line with the recent trend to reduce distribution procedures and distribution time for agricultural food in order to improve food quality, equipments are being developed that could reduce the time needed to detect harmful materials in agricultural food. However, despite the importance, it is difficult to detect in real time such harmful materials of agricultural food such as microorganisms, toxins, and the like, and thus even when a harmful material occurs, it cannot be quickly prevented from spreading, thereby severely damaging consumers.

It is difficult to make an exact real time prediction of harmful materials based on information detected by detection equipments at a base point or fragmentary analyses from information provided as amounts of delivery due to limitation of information. In order to make a more accurate real time prediction of harmful materials, analyses must be made based on not only such detection information and amounts of delivery but also based on harmful material patterns from history data of past to present, current delivery environment (temperature, humidity), real time information widespread in social networks, and other various types of information.

Furthermore, in order to make real time predictions on harmful materials and to prevent the spread of harmful materials, it is required to develop techniques for overall predictions on harmful materials at main base points (distribution phase) of the entire life cycle from production sites to consumers.

### SUMMARY

A purpose of the present disclosure is to predict harmful materials in real time based on big data when a harmful material occurs at a distribution base point using various harmful material information, that is, to provide a real time harmful material prediction service based on various information such as information detected by detecting equipments at a base point or information provided as amounts of delivery, harmful material patterns from history data of past to present, delivery environment (temperature, humidity), and real time information widespread in social networks, in order to quickly and precisely prevent the spread of harmful materials.

According to an embodiment of the present invention, there is provided a harmful material prediction system including a harmful material feature collecting unit configured to collect harmful material features of food; a preprocessing unit configured to preprocess the harmful material features collected and generate harmful material information of an analyzable format; and a Hadoop-based dispersed cluster configured to generate a similarity group where similarity base points are grouped, based on a correlation per variable included in the harmful material information.

In the embodiment, the Hadoop-based dispersed cluster may include a similarity measuring unit per variable configured to generate a similarity matrix per variable based on the correlation per variable included in the harmful material information; a final similarity measuring unit configured to generate a final similarity matrix based on the similarity material per variable; and a similarity group computing unit configured to generate the similarity group where similarity base points are grouped, based on the final similarity matrix.

In the embodiment, the harmful material features may include at least one of a human big data, system big data and social big data, and the harmful material feature collecting unit collects the human big data based on a sqoop, collects the system big data based on a flume, and collects the social big data based on a crawler.

In the embodiment, the human big data may include an institution generated data, the system big data comprises data generated from a detection equipment, and the social big data comprises text data generated through social media.

In the embodiment, the Hadoop-based dispersed cluster may include a plurality of storages that store the harmful material information.

According to another embodiment of the present invention, there is provided a harmful material prediction method including collecting harmful material features of food; preprocessing the collected harmful material features, and generating harmful material information of an analyzable format; and generating, by a Hadoop-based dispersed cluster, a similarity group where similarity base points are grouped, based on a correlation per variable included in the harmful material information.

In the embodiment, the generating a similarity group where similarity base points are grouped based on a correlation per variable included in the harmful material information may include storing the harmful material information in the Hadoop-based dispersed cluster; analyzing, by the Hadoop-based dispersed cluster, the correlation per variable included in the harmful material information and generating a similarity matrix per variable; generating, by the Hadoop-based dispersed cluster, a final similarity matrix based on the similarity matrix per variable; and generating a similarity group where similarity base points are grouped based on the final similarity matrix.

In the embodiment, the storing the harmful material information in the Hadoop-based dispersed cluster may store the harmful material information repeatedly in at least two slave nodes included in the Hadoop-based dispersed cluster.

In the embodiment, the harmful material prediction method may further include visualizing and displaying the generated similarity group.

In the embodiment, the harmful material features may include at least one of human big data, system big data, and social big data, and the collecting harmful material features of food may collect the human big data based on a sqoop, collect the system big data based on a flume, and collect the social big data based on a crawler.

According to the technique of the present disclosure, it is possible to provide a real time harmful material prediction service based on various information such as information detected by detecting equipments at a base point or information provided as amounts of delivery, harmful material patterns based on history data from past to present, delivery environment (temperature, humidity), and real time information widespread in social networks, in order to quickly and precisely prevent the spread of harmful materials.

### BRIEF DESCRIPTION OF THE DRAWINGS

The above and other features and advantages of the present invention will become more apparent to those of ordinary skill in the art by describing in detail embodiments with reference to the attached drawings in which:

FIG. 1 is an exemplary view illustrating a concept of big data being collected at every phase of a distribution process;

FIG. 2 is a block diagram illustrating a system for predicting harmful materials according to an embodiment of the present disclosure;

FIG. 3 is a flowchart illustrating a method for predicting harmful materials according to another embodiment of the present disclosure;

FIG. 4 is a view illustrating information on harmful materials being collected during a distribution process; and

FIG. 5 is a block diagram illustrating an example of a system for predicting harmful materials according to an embodiment of the present disclosure.

### DETAILED DESCRIPTION

Hereinafter, embodiments will be described in greater detail with reference to the accompanying drawings. Embodiments are described herein with reference to cross-sectional illustrates that are schematic illustrations of embodiments (and intermediate structures). As such, variations from the shapes of the illustrations as a result, for example, of manufacturing techniques and/or tolerances, are to be expected. Thus, embodiments should not be construed as limited to the particular shapes of regions illustrated herein but may include deviations in shapes that result, for example, from manufacturing. In the drawings, lengths and sizes of layers and regions may be exaggerated for clarity. Like reference numerals in the drawings denote like elements.

Terms such as ‘first’ and ‘second’ may be used to describe various components, but they should not limit the various components. Those terms are only used for the purpose of differentiating a component from other components. For example, a first component may be referred to as a second component, and a second component may be referred to as a first component and so forth without departing from the spirit and scope of the present invention. Furthermore, ‘and/or’ may include any one of or a combination of the components mentioned.

Furthermore, ‘connected/accessed’ represents that one component is directly connected or accessed to another component or indirectly connected or accessed through another component.

In this specification, a singular form may include a plural form as long as it is not specifically mentioned in a sentence. Furthermore, ‘include/comprise’ or ‘including/comprising’ used in the specification represents that one or more components, steps, operations, and elements exist or are added.

Furthermore, unless defined otherwise, all the terms used in this specification including technical and scientific terms have the same meanings as would be generally understood by those skilled in the related art. The terms defined in generally used dictionaries should be construed as having the same meanings as would be construed in the context of the related art, and unless clearly defined otherwise in this specification, should not be construed as having idealistic or overly formal meanings.

The present disclosure relates to a technique of predicting the spread of harmful materials by acknowledging in real time the situations of harmful materials in agricultural food using various standardized, semi-standardized, and unstandardized information on distribution of agricultural food. Big data is defined in aspects of 4V (Volume, Variety, Velocity, Value) in that it is generally of large volume, and that it realizes excellent value creation and provides important prediction analyses through real time processing of various data.

The present disclosure analyses big data that is big not only in size of the data but also in the variety of the data. In order to predict harmful materials, the present disclosure predicts situations by analyzing patterns of harmful materials based on detection information detected by detecting equipments and history data from the past to present, and uses information from which consumer sentiment may be acknowledged such as SNS (Social Network Service) and the like. As such, in the present disclosure, a prediction model is developed by utilizing not only standardized data generally applied based on big data but also semi-standardized and unstandardized type materials and by obtaining various sources of information that may be used in analyzing. The present disclosure collects in real time the information on direct features (detection information), indirect features (temperature, humidity), and SNS of harmful materials from production sites to consumers, and then stores and processes the collected information in a Hadoop-based dispersed cluster. Analyses are made on correlations between the features of the harmful materials stored in the cluster, important features that would affect the prediction are extracted, similarities are measured for each feature, and then based on the similarities measured for each feature, an overall similarity is derived. Then, similarity groups are optimized so as to derive a result of prediction on the harmful materials.

More specifically, in order to make a precise prediction on food harmful materials, various types of real time information on harmful materials are collected. Information collecting is made from the entire sections of distribution including production sites, storages, processing companies, logistics centers, retail stores, and consumers. The information being collected may include system big data, social big data, and human big data depending on the characteristics of the information. System big data may include detection information that shows whether or not toxins and the like occurred through detection equipments (direct features), indirect features such as temperature and humidity, and periodic events and the like. Such system big data may be data that occurs in real time. For example, temperature information of a certain base point or temperature information of a delivery vehicle and the like may occur in real time and be collected. Social big data may include text information widespread through SNS. For example, in a case where a text stating that food poison occurred after taking a certain food in a certain area is spread through SNS, text information containing those keywords (food, food poison, area name and the like) may be included in the social big data. Social media may include twitter, facebook, blog, and internet news and the like. Human big data may include data generated by an institution. For example, human big data may include weather forecast data generated by a meteorological agency, and information on safety of agricultural products generated by the Agency of Education, Promotion and Information Service in Food, Agricultural, Forestry and Fisheries. Such human big data may be data that may be accumulated in a database.

These types of various data may be configured to have a certain attribute depending on the characteristics of each type of information. In the case of base point information, temperature of the base point, humidity of the base point, amount of items being delivered from the base point, and words related to harmful materials widespread through twitters near the base point may be included as attributes

Furthermore, in the case of a distribution vehicle between base points, when passing each base point, not only information on the temperature of the distribution vehicle at each base point, but also information on the temperature and humidity and the like of inside a distribution vehicle may be provided during passing through a distribution section.

SNS information is social network service information. For example, in the case of twitter, a keyword of a harmful material is predefined, and such a keyword is searched in short twitter sentences. By a result of the search, it is possible to obtain information on the harmful material of the social network that occurred near each base point.

Big data that is collected in a Hadoop-based dispersed cluster may have a different tool depending on the various data characteristics (features) of the big data. Human big data is existing accumulated data, and existing DB contents may be stored in the Hadoop-based dispersed cluster. System big data is data that occurs in real time that may be stored in the Hadoop-based dispersed cluster through a flume. Social big data, that is text information such as short sentences or news of a keyword in SNS and twitter may be stored in the Hadoop-based dispersed cluster through a crawler. The collected information is stored in the Hadoop-based dispersed cluster consisting of a master node and a plurality of slave nodes by an internal RPC-remote procedure call.

The various types of information dispersedly stored in the Hadoop cluster are executed by an execution task dispersedly stored in the slave node to be analyzed. The system big data, human big data, and social big data collected may be in a format or may include contents that cannot be used easily in analyses. Thus, data pre-processing must be performed to refine those contents.

Furthermore, analyses using various types of information may be more accurate than analyses using one piece of information, but in order to perform a meaningful analysis, it is important to analyze the correlations between elements, that are various types of information. If the elements (information) subject to analyses are correlated to one another, it is needless to consider those elements that are correlated to one another as part of the overall elements for analyses. In such cases, the analyzing becomes more complicated, and takes more processing time. Furthermore, even though the accuracy increases, variables of a certain number or more are not desirable. For this purpose, correlations between the elements are analyzed, and then important elements, that is, variables are extracted. As such, when the correlation between the temperature and humidity has similarity, they are integrated and used as one variable.

In the actual analyzing, similarity is calculated for each base point for each important variable extracted. A similarity matrix for each variable is generated based on the similarity result of each base point for each variable calculated as mentioned above. Furthermore, a final similarity matrix having the similarity matrix for each variable as meta data may be generated.

For example, a similarity matrix for each variable consisting of standardized similarity scores of the temperature variable, similarity scores by the humidity variable, and similarity scores by the distribution amount variable is generated. Then, the final similarity matrix is generated, and based on the final similarity matrix, a similarity group is generated. As the similarity is calculated repeatedly, the final similarity group is optimized.

Such an analysis means in a case where the temperature is or above a certain value, and the base point is in a certain area, when a harmful material occurs in that area, there is possibility the harmful material may occur in other areas (base points) grouped in the same group as that area (base point) in the similarity group.

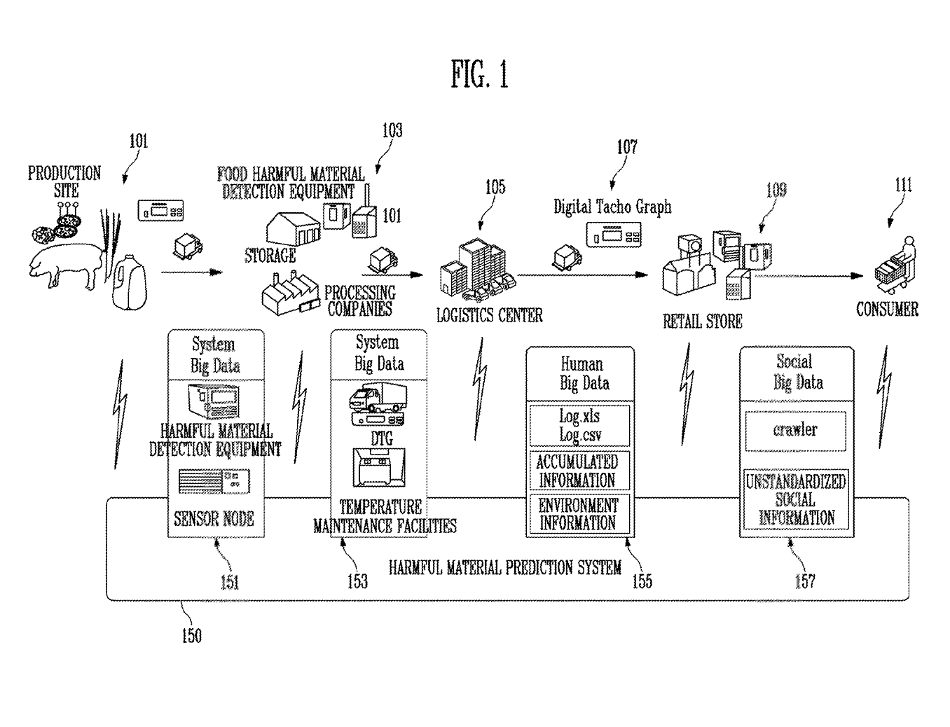

FIG. 1 is an exemplary view illustrating a concept of big data being collected at every phase of a distribution process.

In FIG. 1, a procedure is illustrated where large volume harmful material features are being collected during a process where food is distributed from a production site 101 to consumers 111. That is, the concept of collecting big data on the overall food life cycle is illustrated.

According to the present disclosure, for a precise prediction on food harmful materials, various harmful material features may be collected in real time by a harmful material prediction system 150. The food harmful material features are collected in the overall distribution sections from the food production site 101, storage and processing companies 103, logistics center 105, retail stores 109, and consumers 111. The harmful materials at the production site may be collected by harmful material detection equipments such as a sensor node and GW and the like. The harmful material features collected being collected by the detection equipment may be system big data 151. The harmful materials at the storage and processing companies 103 may also be collected as system big data 153 by a digital tacho graph (DTG) of a vehicle that delivers food from the production site 101 to the storage and processing companies 103. Furthermore, human big data 155 that includes scale information and environment information, and social big data 157 that includes unstandardized social information may be collected by the harmful material prediction system 155. As aforementioned, the system big data 151, 153 may include the detection information, temperature, humidity and the like of the harmful materials. The social big data 155 may include SNS information such as information on twitter, facebook, and blogs, and news. The human big data 157 may include data generated by an institution. For example, the human big data 157 may include weather forecast data generated by a meteorological agency, and information on safety of agricultural products generated by the Agency of Education, Promotion and Information Service in Food, Agricultural, Forestry and Fisheries.

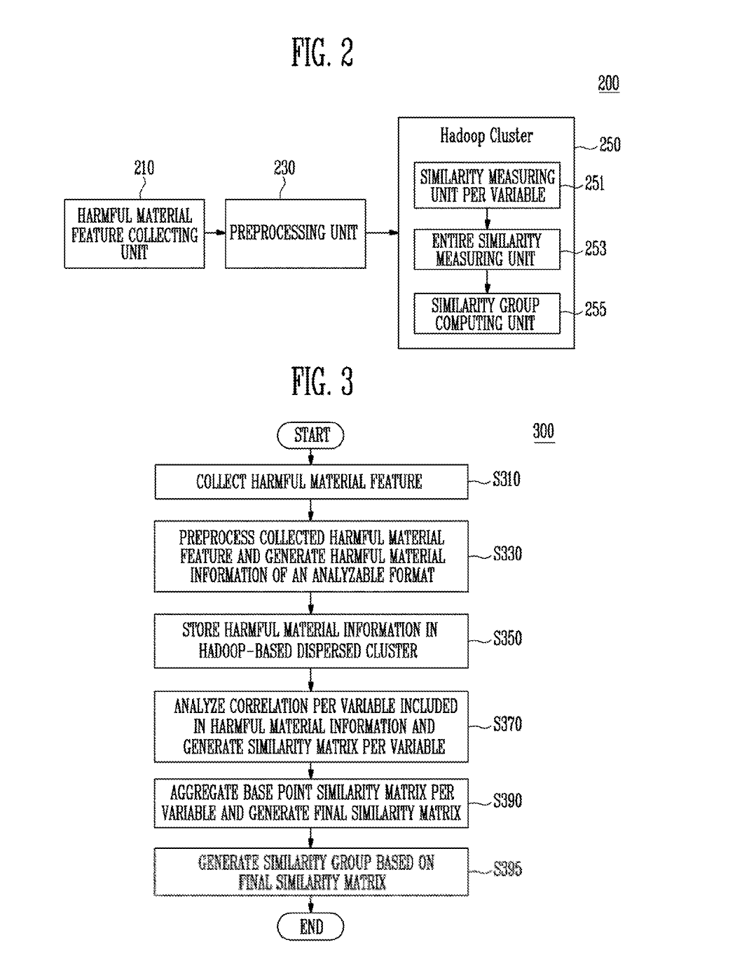

FIG. 2 is a block diagram illustrating a system for predicting harmful materials according to an embodiment of the present disclosure.

Referring to FIG. 2, the harmful material prediction system according to the embodiment of the present disclosure 200 includes a harmful material feature collecting unit 210, preprocessing unit 230, and Hadoop-based dispersed cluster 250. The harmful material feature collecting unit 210 collects harmful material features of food. The preprocessing unit 230 preprocesses the collected harmful features and generates harmful material information in a format that is analyzable. The Hadoop-based dispersed cluster 250 generates a similarity group where similarity base points are grouped based on a correlation per variable included in the harmful material information.

As illustrated in FIG. 1, in the harmful material feature collecting unit 210, information on the temperature and humidity may be collected through a sensor at each phase of distribution. Such information is system big data where direct features showing whether or not harmful materials are detected, temperature information, and humidity information may be collected through a detection equipment, sensor node, Digital Tacho Graph (DTG), thermometer and hygrometer and the like. In an embodiment, these types of the information may be collected by a flume.

Furthermore, the harmful material feature collecting unit 210 may collect data generated by an institution such as the Agency of Education, Promotion and Information Service in Food, Agricultural, Forestry and Fisheries or a meteorological agency. This may be achieved by collecting the database information that each institution periodically or temporarily updates. Such information is human big data, and the harmful material collecting unit 210 may collect the information by a Sqoop.

Furthermore, the harmful material feature collecting unit 210 may collect social big data that includes twitter, facebook, blog, and internet news and the like. Such social big data is mostly text information that may be collected by a crawler.

The collected harmful material features may be in formats that may be directly used in data analyses, or in formats that cannot be easily applied to analyses. The preprocessing unit 230 analyses the collected harmful material features, and if the features are in formats that may be directly applied to analyses, they are transmitted to the Hadoop-based dispersed cluster 250 without additional processing, whereas if the collected harmful material features need to be modified, they are preprocessed such that harmful material information of an analyzable format is generated. For example, when a text including a related keyword is searched by a crawler, harmful material information may be generated such that a table shows the number of times a text occurred for each keyword. In such a case, the searched texts are raw data that correspond to harmful material features of before preprocessing is made. Furthermore, the table that includes the number of times a text occurred for each keyword may be preprocessed information corresponding to harmful material information.

Furthermore, the preprocessing unit 230 may analyze correlations between variables (temperature, humidity and the like) according to the characteristics of the collected harmful material features, and extract important variables. Highly correlated variables may be integrated. For example, if temperature and humidity have a high correlation to each other, processing these two variables separately may be repetition of operation. Therefore, it is possible to integrate the two variables into one important variable and reduce the amount to be analyzed during processing. Whether or not variables should be integrated depends on whether a correlation between the two variables is higher than a predetermined critical value. It is also possible to select one of the two variables when integrating the two variables. For example, in a case where it is has been decided to integrate temperature and humidity since the correlation between the two is high enough, only the temperature may be selected. In such a case, the harmful material features that include only humidity may not be transmitted to the Hadoop-based dispersed cluster 250 whereas the harmful material features that include only temperature are transmitted to the Hadoop-based dispersed cluster 250. Whether or not to integrate two variables may be changed anytime depending on the changes in correlation between the two variables as time goes by. For example, in a case where the correlation between two variables that used to be integrated became lower, without being integrated into one important variable, the two variables may be individually transmitted to the Hadoop-based dispersed cluster 250 as separate important variables.

The Hadoop-based dispersed cluster 250 may include a similarity measuring unit per variable 251, final similarity measuring unit 253, and similarity group computing unit 255. The similarity measuring unit per variable 251 may generate a similarity matrix per variable based on a correlation per variable included in the harmful material information. The final similarity measuring unit 253 may generate the final similarity matrix based on the similarity matrix per variable. The similarity group computing unit 255 may generate a similarity group where similarity base points are grouped based on the final similarity matrix.

The similarity measuring unit per variable 251 may analyze a similarity correlation between base points based on numerous variables (temperature, humidity, number of occurrence of twits including a keyword and the like), and generate a similarity matrix. For example, a similarity matrix between base points according to temperature may be calculated as in table 1 below.

TABLE 1

| Temperature | Base point 1 | Base point 2 | Base point 3 | Base point 4 |
|---|---|---|---|---|
| Base point 1 | — | 0.56 | 0.73 | 0.85 |
| Base point 2 | 0.56 | — | 0.63 | 0.44 |
| Base point 3 | 0.73 | 0.63 | — | 0.92 |
| Base point 4 | 0.85 | 0.44 | 0.92 | — |

In table 1 above, looking at the numerous variables (temperature, humidity) related to a certain base point, the closer the number of the base point is to 1, the higher the similarity, and the closer the number of the base point is to 0, the lower the similarity. Base point 3 and base point 4 have a similarity of 0.92, while base point 2 and base point 4 have a similarity of 0.44. This means that the temperature of base point 4 is similar to the temperature of base point 3, and that the temperature of base point 4 and the temperature of base point 2 are relatively different from each other. A similarity of temperature may be calculated in various ways. In a case where a harmful material actually occurred at base point 4, it is possible to assume that the probability a same harmful material will occur is higher at base point 3 which has a high similarity regarding temperature than other base points. Variables such as humidity may also be calculated in a similar way as above and a similarity matrix may be generated.

Furthermore, for example, a similarity matrix regarding a distribution amount of a certain item may be generated based on how much amount of delivery has been stored and released at a certain base point. That is, for each distribution base point, it is possible to convert the number of times of delivery of the distribution base point where a harmful material occurred and the amount of delivery item that the base point has into scores, and then determine that the distribution base point with a high score has a high similarity with the distribution base point where the harmful material occurred.

In the case of SNS information, for a certain base point, it is possible to generate a similarity matrix depending on how much text that includes a predefined keyword has occurred. For example, in the case the type of SNS is twitter, and the keyword is “colon bacillus” and “pork”, it is possible to first generate a matrix of number of twits per base point according to the twits or re-twits that include the keywords near each base point, and then standardize those to generate a similarity matrix.

That is, the similarity measuring unit per variable 251 may generate a similarity matrix per base point for each variable based on the harmful material information on important variables transmitted from the preprocessing unit 230.

The final similarity measuring unit 253 may generate a final similarity matrix based on the generated similarity matrix per variable. Herein, the similarity matrix per variable may be used as meta data. A different weight may be put to the similarity matrix per variable when generating the final similarity matrix depending on its importance. For example, in a case where a harmful material is highly affected by temperature, it is possible to put a high weight to the similarity matrix per variable according to temperature and generate a final similarity matrix. That is, a final similarity matrix may be generated in the format of aggregating a similarity matrix per variable for each important variable. For example, the final similarity matrix may be generated as in table 2 below.

TABLE 2

| Final similarity | Base point 1 | Base point 2 | Base point 3 | Base point 4 |
|---|---|---|---|---|
| Base point 1 | — | 0.85 | 0.23 | 0.47 |
| Base point 2 | 0.85 | — | 0.91 | 0.15 |
| Base point 3 | 0.23 | 0.91 | — | 0.58 |
| Base point 4 | 0.47 | 0.15 | 0.58 | — |

In table 2 above, looking at the numerous variables (temperature, humidity) related to a certain base point, for every base point, a final similarity with another base point is digitized and calculated. The closer the number of the base point is to 1, the higher the similarity, and the closer the number of the base point is to 0, the lower the similarity. For example, in a case where a harmful material actually occurred near base point 2, it is possible to assume that the probability a same harmful material will occur is higher at base point 3 which has a high similarity, 0.91, than other base points. Therefore, it is possible to prevent or respond to harmful materials by quickly performing concentrated quarantine or quality control on items for base point 3 Meanwhile, since base point 1 has a quite high similarity of 0.85, it is possible to prevent or respond to harmful materials for base point 1 secondly after base point 3. Meanwhile, since base point 4 has a relatively low similarity of 0.15, base point 4 may be processed with a low priority when preventing or responding to harmful materials.

The similarity group computing unit 255 may group the base points having high similarity based on the final similarity matrix. Referring to [table 2], since the similarity of base point 1 and base point 2 is high, base point 1 and base point 2 may be grouped into a same group. Furthermore, since the similarity of base point 2 and base point 3 is high, base point 2 and base point 3 may be grouped into a same group. However, since the similarity of base point 1 and base point 3 is low, base point 1 and base point 3 are not grouped into a same group. That is, base points 1, 2, and 3 are not grouped into one group, but rather, base points 1 and 2 are grouped into a first group, and base points 2 and 3 are grouped into a second group. If all three base points have high similarities, the three base points may be grouped into one group. The grouped base points may be used for quick responding when preventing harmful materials in real time. In the above example, in a case where a harmful material occurred in base point 1, it is possible to predict in real time that there is high probability that a harmful material will occur in base point 2 as well. In a case where a harmful material occurred in base point 2, it is possible to predict in real time that there is high probability that the harmful material will occur in base points 1 and 3 as well.

Although not illustrated in FIG. 2, the harmful material prediction system according to an embodiment of the present disclosure may further include a display unit that displays the final similarity matrix generated in the final similarity measuring unit 253 or the similarity group generated in the similarity group computing unit 255. The display unit may intuitively display the final similarity matrix or similarity group, and thus it is possible to respond more quickly when a harmful material occurs.

As aforementioned, according to the harmful material prediction system according to an embodiment of the present disclosure, it is possible to provide a real time harmful material prediction service based on various types of information such as information detected by detecting equipments at a base point or information provided as amounts of delivery, harmful material patterns based on history data from the past to present, delivery environment (temperature, humidity), and real time information widespread in social networks, in order to quickly and precisely prevent the spread of harmful materials.

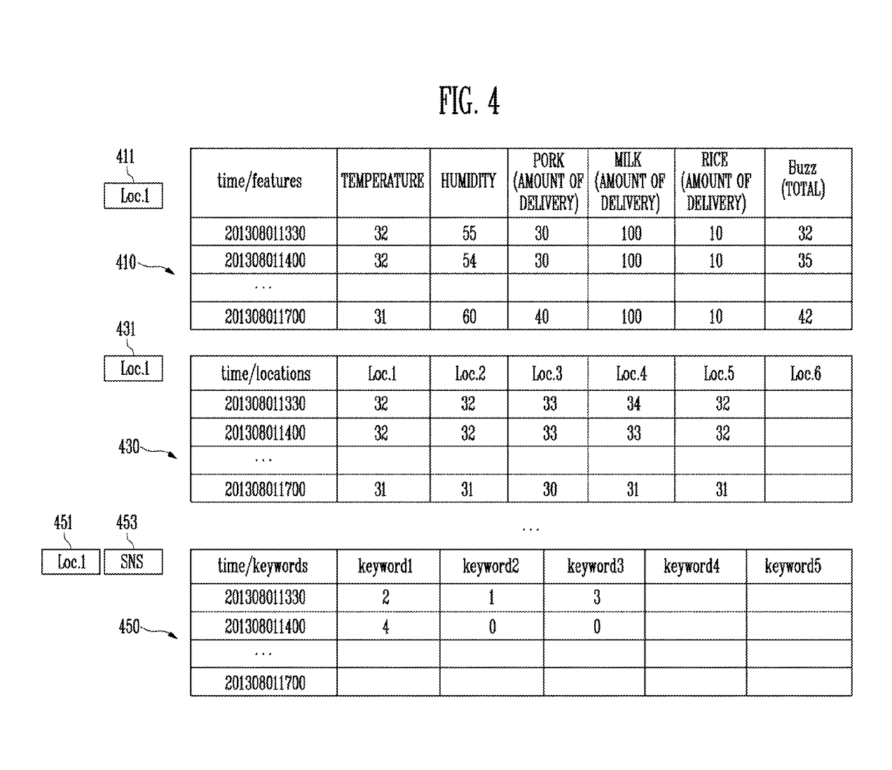

FIG. 3 is a flowchart of a method for predicting harmful materials according to another embodiment of the present disclosure.

Referring to FIG. 3, the harmful material prediction method according to another embodiment of the present disclosure includes collecting a harmful material feature (S310), preprocessing the collected harmful material feature to generate harmful material information of an analyzable format (S330), storing the harmful material information in a Hadoop-based dispersed cluster (S350), analyzing a correlation per variable included in the harmful material information to generate a similarity matrix per variable (S370), aggregating the generated base point similarity matrix to generate a final similarity matrix (S390), and generating a similarity group based on the final similarity matrix (S395). Herein, steps (S350) to steps (S395) may be performed by the Hadoop-based dispersed cluster.

At the step of collecting a harmful material feature (S310), big data including the harmful material feature may be collected in various ways. As illustrated in FIG. 1, information on temperature and humidity may be collected by a sensor and the like at each step of distribution. Such information is system big data that may be collected through a detection equipment, sensor node, Digital Tacho Graph (DTG), thermometer and hygrometer and the like. In an embodiment, such information may be collected by a flume.

Furthermore, at the step of collecting a harmful material feature (S310), data generated by a meteorological agency, and information on safety of agricultural products generated by the Agency of Education, Promotion and Information Service in Food, Agricultural, Forestry and Fisheries may be collected. This may be achieved by collecting the database information that each institution periodically or temporarily updates. Such information is human big data, and the harmful material collecting unit 210 may collect the information by a Sqoop.

Furthermore, at the step of collecting a harmful material feature (S310), social big data that includes SNS information such as information on twitter, facebook, and blogs, and news may be collected. Such social big data is mostly text information that may be collected by a crawler.

At the step of preprocessing the collected harmful material feature to generate harmful material information of an analyzable format (S330), the collected harmful material features are analyzed, and if the features are of a format that may be directly analyzed, the features are transmitted to the Hadoop-based dispersed cluster without processing, and if the features needed to be modified, harmful material information of an analyzable format may be generated and transmitted to the Hadoop-based dispersed cluster. For example, when a text including a related keyword is searched by a crawler, harmful material information may be generated such that a table is generated where the number of times a text occurred for each keyword are aligned. In such a case, the searched texts are raw data that correspond to harmful material features of before preprocessing is made. Furthermore, the table that includes the number of times a text occurred for each keyword may be preprocessed information corresponding to harmful material information.

Furthermore, at step (S330), correlations between variables (temperature, humidity and the like) according to the characteristics of the collected harmful material features may be analyzed, and important variables may be extracted. Highly correlated variables may be integrated. For example, if temperature and humidity have a high correlation to each other, processing these two variables separately may be repetition of operation. Whether or not to integrate two variables may be determined by determining whether or not the correlation between the two variables is higher than a predetermined critical value. When integrating the two variables, it is also possible to select one of the two variables instead. For example, when it has been determined to integrate temperature and humidity since the correlation between the two is high, it is possible to select temperature only. In such a case, a harmful material feature that includes humidity only may not be transmitted to the Hadoop-based dispersed, but a harmful material feature including temperature may be transmitted to the Hadoop-based dispersed cluster. Whether or not to integrate two variables may be changed anytime depending on the change of correlation between the two variables as time goes by. For example, in a case where the correlation between two variables that used to be integrated became lower after a certain time point, without being integrated into one important variable, the two variables may be individually transmitted to the Hadoop-based dispersed cluster as separate important variables.

At the step of storing harmful material information in the Haddop-based dispersed cluster (S350), the harmful material information may be stored in a plurality of storages inside the Hadoop-based dispersed cluster. The Hadoop-based dispersed cluster may include a plurality of storages for storing the harmful material information. As will be explained hereinafter, the Hadoop-based dispersed cluster may include a master node and a plurality of slave nodes. Furthermore, each of the master node and the plurality of slave nodes may include storage so that the harmful material information may be stored in at least some of each storage of the master node and the plurality of slave nodes. Furthermore, the Hadoop-based dispersed cluster may include a plurality of storages of a database format configured separately from the master node and plurality of slave nodes. In such a case, the harmful material information may be stored in at least some of the plurality of storages of a database format. In an embodiment, a same piece of harmful material information may be stored in a plurality of storages repeatedly.

At the step of analyzing a correlation per variable included in the harmful material information to generate a similarity matrix per variable (S370), a similarity correlation between base points may be analyzed based on numerous variables (temperature, humidity, number of occurrence of twits including a keyword and the like) regarding a certain base point to generate a similarity matrix. For example, a similarity matrix between base points regarding temperature may be generated with reference to [table 1] as mentioned above. Furthermore, a similarity matrix regarding the amount of distribution of a certain item, for example, may be generated based on how much delivery amount has been stored and released. In the case of SNS information, a similarity matrix may be generated based on how much text that includes a predefined keyword has occurred regarding a certain base point. That is, at the step (S370), it is possible to generate a similarity matrix per base point regarding each variable based on the harmful material information regarding important variables.

At the step of aggregating the base point similarity matrix per variable generated to generate a final similarity matrix (S390), it is possible to generate a final similarity matrix where all the important variables have been considered using similarity matrix per variable as meta data. A different weight may be put to the similarity matrix per variable when generating the final similarity matrix depending on its importance. For example, in a case where a harmful material is highly affected by temperature, it is possible to put a high weight to the similarity matrix per variable according to temperature and generate a final similarity matrix. That is, the final similarity matrix may be generated in the format of aggregating a similarity matrix per variable for each important variable. For example, the final similarity matrix may be generated as in table 2 below.

At the step of generating a similarity group based on the final similarity matrix (S395), base points having a high similarity may be grouped based on the final similarity matrix.

Although not illustrated in FIG. 3, the harmful material prediction method according to an embodiment of the present disclosure may further include visualizing the generated similarity group and displaying the same. At the step of displaying the similarity group, the similarity group may be displayed intuitively on a display apparatus and the like, and thus it is possible to respond more quickly when a harmful material occurs.

As aforementioned, according to a harmful material prediction method according to another embodiment of the present disclosure, it is possible to provide a real time harmful material prediction service based on various information such as information detected by detecting equipments at a base point or information provided as amounts of delivery, harmful materials patterns based on history data from the past to the present, delivery environments (temperature, humidity), and real time information well known in social networks, in order to quickly and precisely prevent the spread of harmful materials.

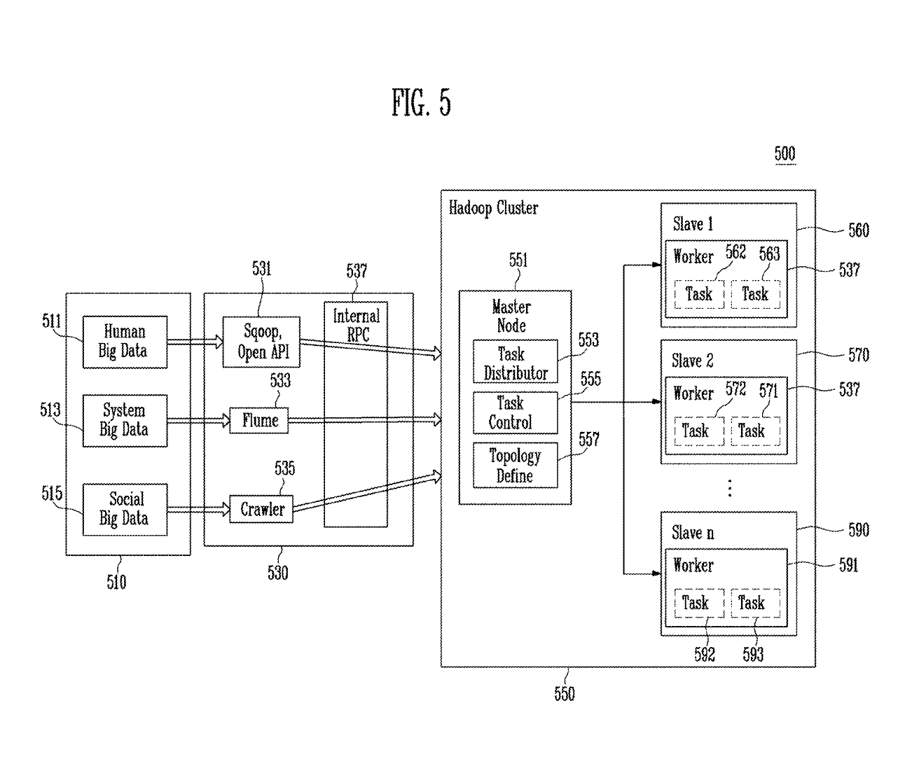

FIG. 4 is a view illustrating information on harmful materials being collected during a distribution process. FIG. 4 is an exemplary illustration of each attribute of various pieces of information being collected in the harmful material prediction system.

Different attributes for harmful material prediction are included. In the case of base point information 410, identification information 411 of a base point (Loc.1) showing which base point it is, and attribute information for each time zone of the base point are displayed. Attributes included in the base point information 410 are temperature, humidity, delivery amount of pork, delivery amount of milk, delivery amount of rice and Buzz number. The Buzz number may be a word related to a harmful material in an SNS text that occurred near the base point (Loc. 1). For example, at the base point information 410, the number 32 of the first line and sixth row may be the number of times a harmful material related keyword such as “food poison, stomachache” occurred in texts near the base point until August 1, 13:30. Various harmful material related keywords may be applied.

In the case of temperature information 430 of a distribution vehicle that runs between base points, when the vehicle runs between a plurality of base points (Loc.1, Loc.2, Loc.3, Loc.4, Loc.5, Loc.6) as distribution sections, a temperature of each time zone at each base point may be included as an attribute. Likewise, humidity information may also be collected or used as a harmful material feature or harmful material information.

The SNS information per base point 450 may include a text that occurred by a social network service. For example, in the case where SNS information 450 includes twitter, social big data may be collected in such a way by predefining a keyword related to a harmful material and then searching the number of twits that include the keyword. FIG. 4 illustrates harmful material features per time zone of SNS 453 related to base point (Loc. 1, 451). Five keywords are defined.

FIG. 5 is a block diagram illustrating an example of a harmful material prediction system according to an embodiment of the present disclosure.

Referring to FIG. 5, the harmful material prediction system according to an embodiment of the present disclosure 500 includes a harmful material feature collecting unit 510, preprocessing unit 530, and Hadoop-based dispersed cluster 550.

The harmful material feature collecting unit 510 may include a human big data collecting unit 511, system big data collecting unit 513, and social big data collecting unit 515. The big data collected may be preprocessed in the preprocessing unit 530 and be stored in the Hadoop-base dispersed cluster 550.

A different tool may be used depending on the characteristics of the data collected. The human big data collected in the human big data collecting unit 511 is an existing accumulated data. It may be stored using the Sqoop 531 so that the information stored in an existing database may be stored in the Hadoop-based dispersed cluster. The system big data collected in the system big data collecting unit 513 is data that occurs in real time that may be stored using the flume 533. The social big data being collected by the social big data collecting unit 515 is text data of short sentences including a keyword in twitter or news that may be collected through a crawler 535. The collected information may be stored in the Hadoop-based dispersed cluster 550 by an internal remote procedure call (RPC) 537. More specifically, the Hadoop-based dispersed cluster 550 may include a master node 551 and a plurality of slave nodes 560, 570, . . . , 590. The master node 551 may include a task distribution unit 553, task control unit 550, and topology defining unit 5570, and the master node 551 may serve to distribute, control, and configure the tasks being distributed in the slave nodes 560, 570, . . . , 590. The plurality of slave nodes 560, 570, . . . , 590 may store the collected harmful material features or harmful material information, and may include processing units 560, 571, . . . , 591 that actually process the tasks 562, 563, 572, 573, . . . , 592, 593 dispersed by the master node 551. Although not illustrated, the Hadoop-based dispersed cluster 550 may include a plurality of storages for storing harmful material information. Each of the master node 551 and the plurality of slave nodes 560, 570, . . . , 590 may be include a storage. There may also be a storage of a database format configured separately from the master node 551 and the plurality of slave nodes 560, 570, . . . , 590.

Herein, it should be understood that each block in the process flowcharts and combinations of the flowcharts may be performed by computer program interactions. These computer program interactions may be mounted onto a general use computer, special use computer, or any other processor of a programmable data processing equipment, and thus the interactions executed by such a computer or any other processor of a programmable data processing equipment generate the tools for performing the functions explained in the flowchart block(s). These computer program interactions may use a computer or a computer oriented to other types of programmable data processing equipment, or may be stored in a computer-readable memory, and thus the interactions that use those computers or the interactions stored in a computer-readable memory may produce an item that include an interaction tool that performs the functions explained in the flowchart block(s). The computer program interactions may be mounted onto a computer or other type of programmable data processing equipment, and thus the interactions where a series of operation steps are performed on a computer or other type of programmable data processing equipment to generate processes executable by a computer or other type of programmable data processing equipment may provide the steps for executing the functions explained in the flowchart block(s).

Furthermore, each block may represent a part of a module that includes one or more executable interactions for executing specified logical function(s), a segment or code. Furthermore, in several alternative embodiments, the functions mentioned in the blocks may be performed in different orders. For example, two blocks illustrated successively may actually be performed simultaneously or in reverse orders.

Herein, the word ‘-unit’ refers to a component of a software or hardware such as FPGA or ASIC, ‘-unit’ performing certain roles. However, ‘-unit’ is not limited to just software and hardware. ‘-unit’ may be configured to be a part of a storage medium for addressing, or may be configured to reproduce one or more processors. Therefore, for example, ‘-unit’ includes software components, object-oriented software components, class components, task components, processes, functions, attributes, procedures, subroutines, segments of program codes, drivers, firmware, microcode, circuit, data, database, data structures, tables, arrays, and variables. The functions provided in the components and ‘-units’ may be combined into a smaller number of components and ‘-units’, or may be further divided into additional components and ‘-units’. Not only that, components and ‘-units’ may be realized to reproduce one or more CPUs in a device or security multimedia card.

In the drawings and specification, there have been disclosed typical exemplary embodiments of the invention, and although specific terms are employed, they are used in a generic and descriptive sense only and not for purposes of limitation. As for the scope of the invention, it is to be set forth in the following claims. Therefore, it will be understood by those of ordinary skill in the art that various changes in form and details may be made therein without departing from the spirit and scope of the present invention as defined by the following claims.

What is claimed is:

1. A harmful material prediction system comprising:
a harmful material feature collecting unit configured to collect harmful material features of food;
a preprocessing unit configured to preprocess the harmful material features collected and generate harmful material information of an analyzable format; and
a Hadoop-based dispersed cluster configured to generate a similarity group where similarity base points are grouped, based on a correlation per variable included in the harmful material information.

2. The system according to claim 1,
wherein the Hadoop-based dispersed cluster comprises:
a similarity measuring unit per variable configured to generate a similarity matrix per variable based on the correlation per variable included in the harmful material information;
a final similarity measuring unit configured to generate a final similarity matrix based on the similarity material per variable; and
a similarity group computing unit configured to generate the similarity group where similarity base points are grouped, based on the final similarity matrix.

3. The system according to claim 1,
wherein the harmful material features comprise at least one of a human big data, system big data and social big data, and
the harmful material feature collecting unit collects the human big data based on a sqoop, collects the system big data based on a flume, and collects the social big data based on a crawler.

4. The system according to claim 3,
wherein the human big data comprises an institution generated data, the system big data comprises data generated from a detection equipment, and the social big data comprises text data generated through social media.

5. The system according to claim 1,
wherein the Hadoop-based dispersed cluster comprises a plurality of storages that store the harmful material information.

6. A harmful material prediction method comprising:
collecting harmful material features of food;
preprocessing the collected harmful material features, and generating harmful material information of an analyzable format; and
generating, by a Hadoop-based dispersed cluster, a similarity group where similarity base points are grouped, based on a correlation per variable included in the harmful material information.

7. The method according to claim 6,
wherein the generating a similarity group where similarity base points are grouped based on a correlation per variable included in the harmful material information comprises:
storing the harmful material information in the Hadoop-based dispersed cluster;
analyzing, by the Hadoop-based dispersed cluster, the correlation per variable included in the harmful material information and generating a similarity matrix per variable;
generating, by the Hadoop-based dispersed cluster, a final similarity matrix based on the similarity matrix per variable; and
generating a similarity group where similarity base points are grouped based on the final similarity matrix.

8. The method according to claim 6,
wherein the storing the harmful material information in the Hadoop-based dispersed cluster stores the harmful material information repeatedly in at least two slave nodes included in the Hadoop-based dispersed cluster.

9. The method according to claim 6,
further comprising visualizing and displaying the generated similarity group.

10. The method according to claim 6,
wherein the harmful material features comprise at least one of human big data, system big data, and social big data, and
the collecting harmful material features of food collects the human big data based on a sqoop, collects the system big data based on a flume, and collects the social big data based on a crawler.
